(19) United States
(12) Patent Application Publication
(10) Pub. No.: US 2013/0022514 A1
(43) Pub. Date: Jan. 24, 2013

(54) APPLIANCE, PARTICULARLY KITCHEN APPLIANCE OR LABORATORY TABLE AND DEODORANT DEVICE

(71) Applicants: Gregor Morfill, Muenchen (DE); Tetsuji Shimizu, Garching (DE); Yang-Fang Li, Garching (DE); Takuya Urayama, Great Britain (GB); Julia Zimmermann, Muenchen (DE)

(72) Inventors: Gregor Morfill, Muenchen (DE); Tetsuji Shimizu, Garching (DE); Yang-Fang Li, Garching (DE); Takuya Urayama, Great Britain (GB); Julia Zimmermann, Muenchen (DE)

(73) Assignees: MAX-PLANCK-GESELLSCHAFT ZUR FOERDERUNG DER WISSENSCHAFTEN E.V., Muenchen (DE); ADTEC EUROPE LTD., Middlesex (GB)

(21) Appl. No.: 13/583,874

(22) PCT Filed: Mar. 10, 2011
(86) PCT No.: PCT/EP11/01177

§ 371 Date: Oct. 5, 2012

(30) Foreign Application Priority Data
Mar. 10, 2010 (EP) PCT/EP2010/001491
May 19, 2010 (EP) 10005236.4

(51) Int. Cl. A61L 2/14

(52) U.S. Cl. 422/186.21

(57) ABSTRACT

The invention relates to an appliance (18), particularly kitchen appliance or laboratory table, for at least partially disinfecting/sterilising a contaminated surface (21), wherein the appliance (18) comprises an integrated plasma source for at least partially disinfecting/sterilising the surface by generating a non-thermal atmospheric plasma on the surface thereby reducing the concentration of pathogenic germs on the surface.

### FIELD OF THE INVENTION

The invention relates to an appliance, particularly a kitchen appliance or a laboratory table, for at least partially sterilizing and/or disinfecting and/or decontaminating a contaminated surface.

### BACKGROUND OF THE INVENTION

The use of non-equilibrium plasmas (often referred to as non-thermal plasmas or low-temperature plasmas) for the in vivo sterilization of wounds is disclosed, for example, in U.S. Pat. No. 7,683,342 B2. However, the plasma source disclosed in this patent is not suitable for the regular sterilization of surfaces of appliances, e.g. kitchen appliances or laboratory tables, under normal operating conditions of the appliance, i.e. during daily use of the appliance.

### SUMMARY OF THE INVENTION

It is therefore an object of the invention to improve the disinfection and/or disinfection and/or decontamination of surfaces of appliances, particularly kitchen appliances or laboratory tables.

This problem is solved by the idea to integrate a plasma source into an appliance, e.g. kitchen appliance or a laboratory table, wherein the integrated plasma source at least partially disinfects/sterilizes the surface by generating a non-thermal plasma on the surface thereby reducing the concentration of pathogenic germs on the surface.

In a preferred embodiment of the invention, the surface to be disinfected/sterilized is a surface of the appliance which is contaminated during use of the appliance. For example, kitchen appliances, e.g. work benches, generally comprise work plates or cutting boards, which are contaminated during the preparation of food. In other words, the surface to be disinfected/sterilized is preferably a part of the appliance which also includes the plasma source.

However, it is alternatively possible that the surface to be disinfected/sterilized is separated from the appliance including the plasma source. For example, the invention also encompasses a deodorant device for deodorizing a body surface, particularly in the form of a roll-on applicator or a spray device. In this embodiment, the plasma source is integrated into the deodorant device while the surface to be disinfected/sterilized is a body surface which is separated from the deodorant device.

In a preferred embodiment of the invention, the plasma source is a surface micro-discharge plasma source comprising several electrodes, wherein the surface micro-discharge plasma source generates micro-discharges on the surface of the plasma source. The basic principles of high-pressure plasma micro-discharges are explained, for example, in Hippler/Kersten/Schmidt/Schoenbach: “Low temperature plasmas”, Second Edition, Wiley Publishing House, Chapter 17. Therefore, reference is made to the afore-mentioned publication with regard to the basic principles of surface micro-discharge plasma sources, so that the aforementioned publication is incorporated by reference herein. However, it should be briefly mentioned that the surface micro-discharge plasma source comprises several electrodes which are spaced apart.

It should further be mentioned that there can be a uniform distance between the adjacent electrodes of different polarity. However, it is alternatively possible that there is spatially variable distance between the adjacent electrodes of different polarity.

Further, it should be noted that the surface micro-discharge plasma source is preferably embedded into the surface of the appliance, so that the non-thermal plasma is generated on top of the surface of the appliance. For example, the plasma source can be embedded in the surface of a work plate of a kitchen table so that the low-temperature plasma is generated on the surface of the work plate of the kitchen table thereby at least partially sterilizing the surface of the work plate.

It should further be noted that the embedded plasma source is preferably embedded in such a way that it is substantially flush with the surface of the appliance. Therefore, the plasma source preferably comprises a substantially plane surface which is flush with the plane surface of the appliance, e.g. a work plate of a kitchen table.

However, the invention is not restricted to appliances comprising a plane plasma source. It is also possible that the electrodes of the surface micro-discharge plasma source have a shape which resembles the shape of the surface of the appliance. For example, the plasma source can be integrated in a curved surface of the appliance so that the invention does not restrict the freedom of design of the appliance.

Further, there is variety of different arrangements of the electrodes of the surface micro-discharge plasma source.

In one embodiment of the invention, the electrodes of the surface micro-discharge plasma source are arranged in the same plane. For example, the electrodes can be finger-shaped intertwining each other from opposite directions. In another embodiment, the electrodes are spiral-shaped intertwining each other. Further, the electrodes can comprise interlocking branches or kinks.

In another embodiment of the invention, the electrodes are accessed sequentially by switching of the grounded part with a “cycling frequency” f_{C}, thus enabling a propagating plasma source across the device. For instance, a “switched self sterilizing surface device” (100×50 cm²) with parallel electrodes (see FIG. 3C) separated by 5 mm operated with a cycling frequency of 1 Hz would have plasma production along a strip of length 50 cm for a time tₚ=10 ms. For a HV frequency f_{HV}=2 kHz this would imply f_{HV}·tₚ 20 bursts of surface micro discharges, enough to start the ion-molecule reaction chain. The plasma afterglow lasts for more than 1 s, so that the next cycle would enhance the local plasma chemistry. Such a device (100×50 cm²) could be operated with a power of 25 W.

In another embodiment of the invention, the electrodes of the micro-discharge plasma source are not arranged in the same plane but in separate adjacent electrode layers, wherein each of the electrode layers is preferably planar and the separate electrode layers are preferably arranged coplanar relative to each other.

Moreover, it should be noted that the electrode arrangement of the plasma source is freely scaleable.

Further, it should be mentioned that the appliance according to the invention is preferably water-proof, dust-proof, air-born particles proof and/or easy to clean. This is particularly advantageous in case of a kitchen table comprising an integrated plasma source for disinfecting the work-plate of the kitchen table.

Moreover, the surface of the appliance comprising the integrated plasma source preferably consists of a corrosion resistant material, particularly ceramics, glass or glass-ceramics.

It has already been mentioned that the appliance according to the invention can be a work plate, particularly on a kitchen table or on a laboratory table, or a cutting board for cutting objects, particularly food stuffs.

However, the plasma source according to the invention can alternatively be integrated into a handle, particularly a door handle, wherein the integrated plasma source sterilizes the surface of the handle.

In another embodiment of the invention, the appliance is a bathroom equipment, particularly a toilet seat, comprising an integrated plasma source for sterilizing the surface of the bathroom equipment.

Further, it has already been mentioned that the invention also encompasses a deodorant device for deodorizing a body surface particularly in the form of a roll-on applicator or a spray device. In this embodiment, the deodorant device comprises an integrated plasma source applying a non-thermal plasma to the body surface which is to be deodorized.

Another application of the invention is the sterilization of a moving handrail of an escalator or a moving walkway. In this embodiment, the plasma source can be arranged stationary close to the surface of the handrail, so that the non-thermal plasma generated by the plasma source at least partially disinfects/sterilizes the surface of the handrail. Alternatively, the plasma source can also be integrated into the moving handrail, so that the plasma source moves with the hand-rail.

Further, the invention also encompasses an appliance in the form of gym equipment, particularly in the form of a bench or a seat of a training machine. In this embodiment, the plasma source is integrated into the gym equipment thereby sterilizing the bacteria produced by the sweat.

It should also be noted that the power supply of the integrated plasma source can be provided wireless by an integrated battery which allows a mobile use of the device. Alternatively, the power supply of the plasma source can be provided by connection to the general mains.

Further, the invention encompasses also a battery operated device for reducing itching caused by insect bite, particularly in the form of a stick comprising the plasma source. In this embodiment, the battery operated device applies a non-thermal plasma to the skin surface at an insect bite thereby reducing itching.

Further, the invention is also suitable for protection against athletes' foot and other fungal deceases, particularly in damp environments, particularly swimming pools and saunas.

Moreover, the invention is suitable for the disinfection of baby bottles, pacifiers, toys, dentures, tooth brushes, razors, shavers, combs or hair brushes.

Another possible application of the invention is the use of a non-thermal plasma in a dishwasher or a dryer for sterilizing the dishes in the dishwasher or dryer.

Further, the concept of the invention can be applied in devices for disinfection of medical equipment or in the food industry for disinfecting objects.

In another embodiment, the appliance according to the invention comprises a conveyor belt, wherein the plasma source is arranged in the vicinity of the conveyor belt so that the plasma sterilizes objects conveyed on the conveyor belt. For example, the plasma source can be arranged beneath the conveyor belt so that the plasma is applied through the belt, which therefore has to be permeable for the plasma. Alternatively, the plasma source can be arranged stationary above or in the vicinity of the conveyor belt so that the plasma generated by the plasma source reaches the objects on the conveyor belt. Further, it is alternatively possible to integrate the plasma source into the conveyor belt.

It has already been mentioned that the invention also encompasses a deodorant device for deodorizing a body surface by applying a non-thermal plasma. In one embodiment, the deodorant device of the invention resembles the design of conventional deodorant devices comprising a rotatable ball. In this embodiment, the plasma source can be integrated into the rotatable ball, wherein the plasma source generates the plasma on the surface of the rotatable ball. Alternatively, the plasma source can be arranged stationary within the housing of the deodorant device but outside the rotatable ball.

In a preferred embodiment of the deodorant device according to the invention, the deodorant device additionally comprises an applicator for applying a deodorant to the skin surface wherein the agent applied to the skin surface interacts with the non-thermal plasma thereby improving the disinfecting/sterilizing effect of the non-thermal plasma. In other words, the non-thermal plasma applied by the plasma source and the chemical agent applied by the applicator interact with each other so that the disinfecting/sterilizing effect is enhanced by the interaction between the chemical agent and the non-thermal plasma.

In another embodiment, the deodorant device does not comprise any moveable parts and relies solely on the disinfecting/sterilizing effect of the plasma.

It should also be noted that the term “pathogenic germs” as used in this description encompasses bacteria, spores, viruses, fungi, prions, micro organisms and bio-films comprising any of the aforementioned pathogenic germs.

The invention and its particular features and advantages will become more apparent from the following detailed description considered with reference to the accompanying drawings.

### BRIEF DESCRIPTION OF THE DRAWINGS

FIG. 1 shows an exploded perspective view of a plasma source according to the invention, which can be integrated into an appliance, e.g. a kitchen table.

FIG. 2 shows a perspective view of the plasma source according to FIG. 1.

FIGS. 3A-3I show different embodiments and views of a plasma source according to the invention, including the “switched self sterilizing surface” in FIG. 3C, wherein switches are closed and opened sequentially at a rate f_{C}/n, with n being the number of grounded electrodes.

FIG. 4 shows a perspective view of a kitchen block comprising a self-sterilizing work plate.

FIG. 5 shows a perspective view of a laboratory table comprising a self-sterilizing work plate.

FIG. 6 shows a perspective view of a toilet seat comprising an integrated plasma source for sterilizing the toilet seat.

FIG. 7A shows a schematic view of an escalator comprising a plasma source for sterilizing the moving handrail of the escalator.

FIG. 7B shows a modification of the embodiment of FIG. 7A wherein the plasma source is integrated into the moving handrail.

FIG. 8 shows a schematic view of the deodorant device comprising an integrated plasma source.

FIG. 9 shows a modification of the embodiment of FIG. 8 additionally comprising nozzles for applying a chemical agent onto the body surface.

FIG. 10 is a modification of the embodiment of FIG. 8 comprising a rotatable ball wherein the plasma source is integrated into the rotatable ball.

FIG. 11 shows a side view of another embodiment of a deodorant device.

FIG. 12 shows a front view of the deodorant device depicted in FIG. 11.

FIG. 13 shows a longitudinal cut through the front end of the deodorant device according to FIGS. 11 and 12.

FIG. 14 shows a perspective view of another example of a deodorant device comprising an integrated plasma source.

FIG. 15 shows a schematic view of a washing machine comprising an integrated plasma source.

FIG. 16 shows a simplified side view of a conveyor comprising a plasma source for sterilizing objects on the conveyor.

FIG. 17 is a diagram showing the switching of the plasma source in the appliance according to the invention.

FIG. 18 shows a schematic cross section through a container.

FIG. 19 shows a top view onto a lid 89 of the container depicted in FIG. 18.

FIG. 20 shows an enlarged cross section through the lid 89.

FIG. 21 shows a top view of a counter electrode 91 of the container shown in FIG. 18.

FIG. 22 shows a lid (left-hand) and a counter electrode (right-hand) which are used in case the lid 89 is soft and bendable.

FIG. 23 shows another embodiment of a lid (left-hand) and a counter electrode (right-hand).

### DETAILED DESCRIPTION OF THE DRAWINGS

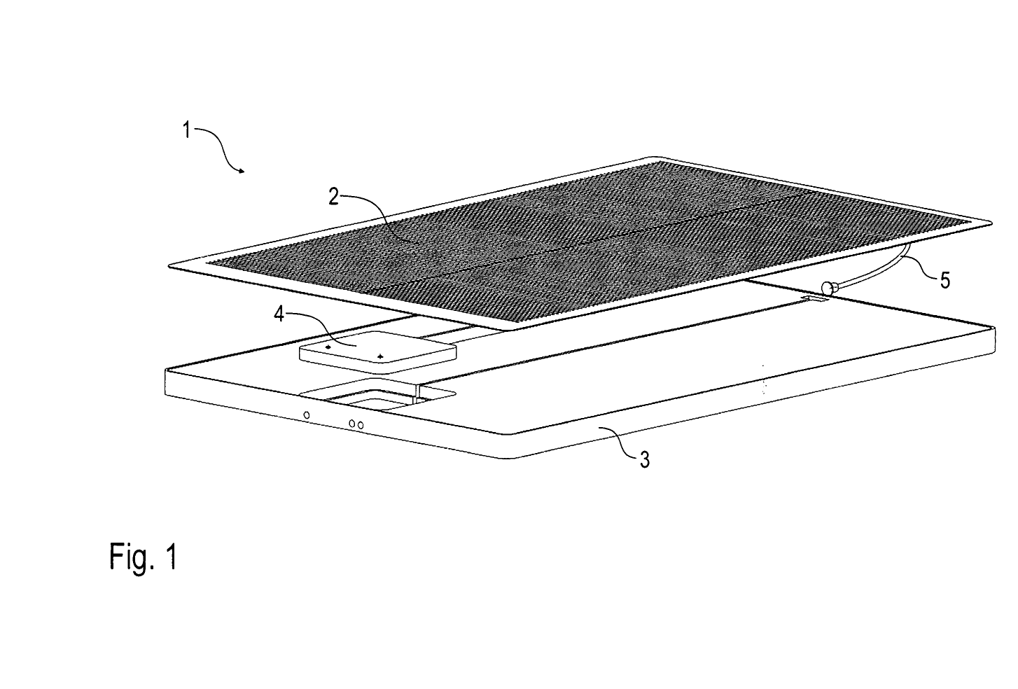

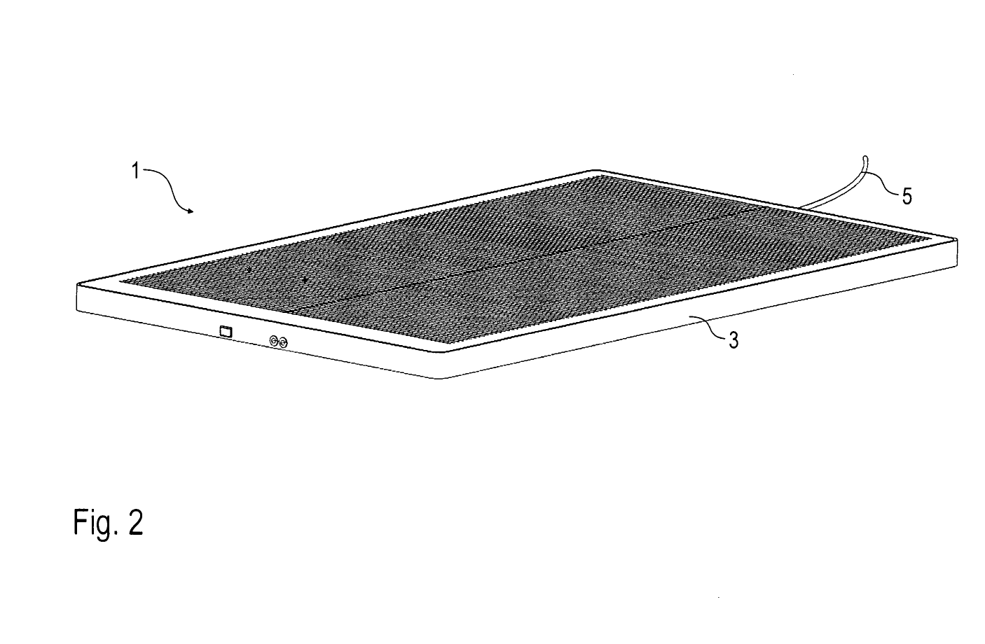

FIGS. 1 and 2 illustrate an embodiment of a plasma source 1 which can be integrated into an appliance, e.g. a work plate of a kitchen table, which will be described in more detail later.

The plasma source 1 comprises a flat and planar electrode arrangement 2, a housing 3, a driver circuit 4 for driving the electrode arrangement 2 and a connection cable 5 for connecting the plasma source 1 to mains.

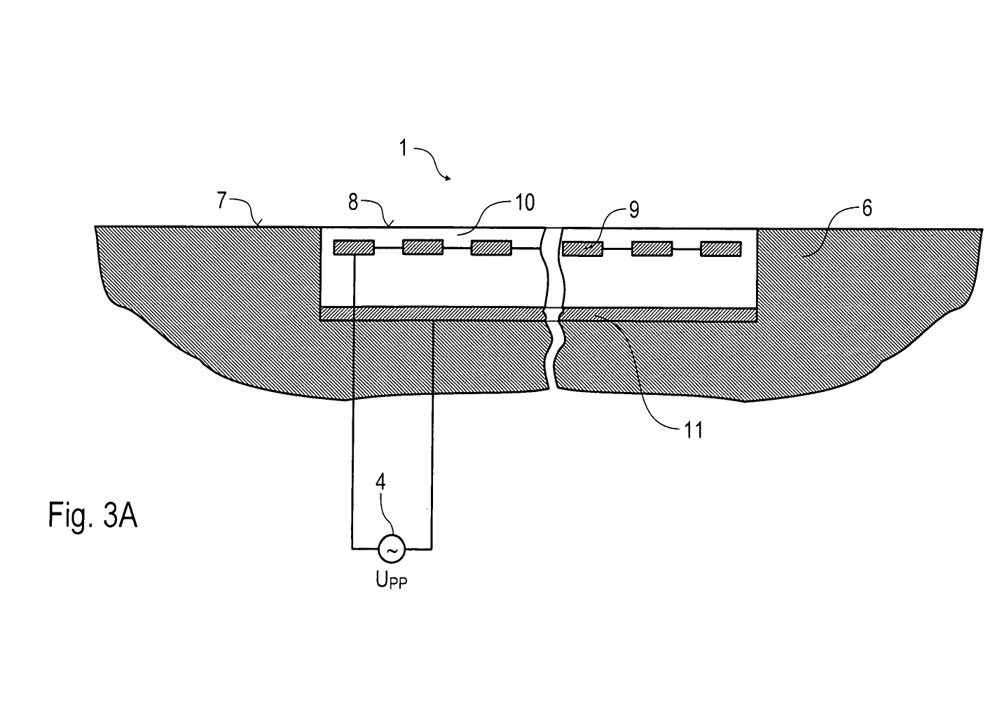

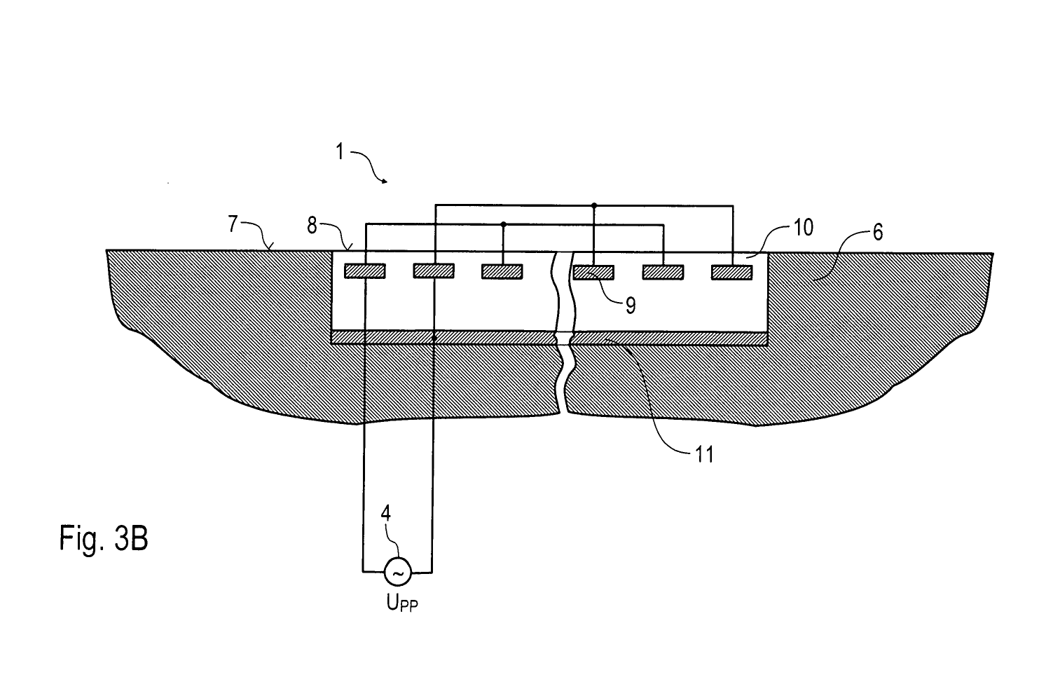

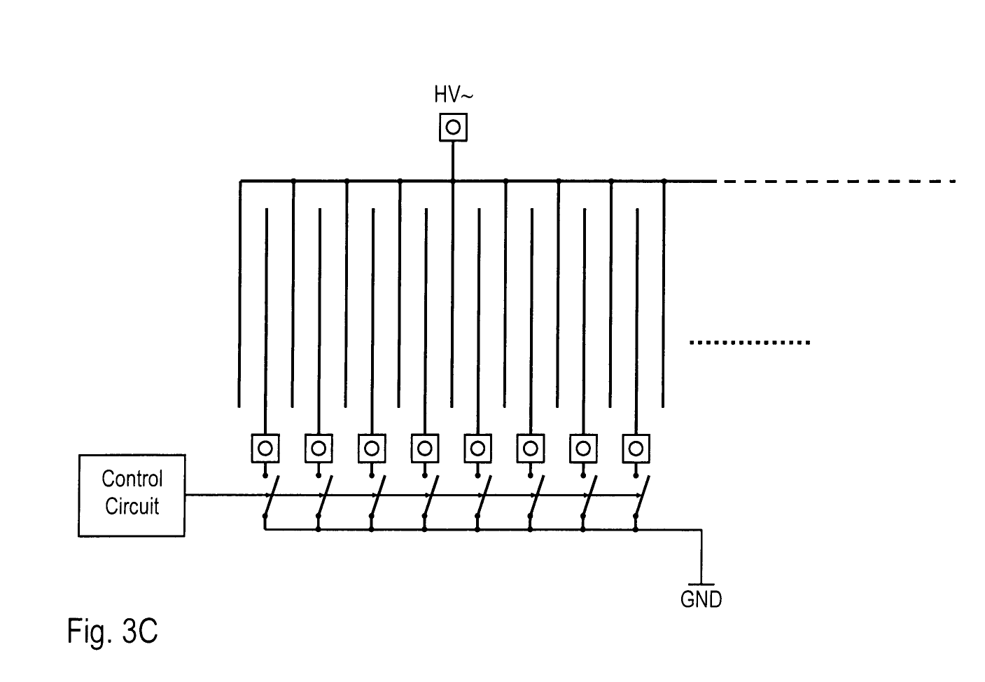

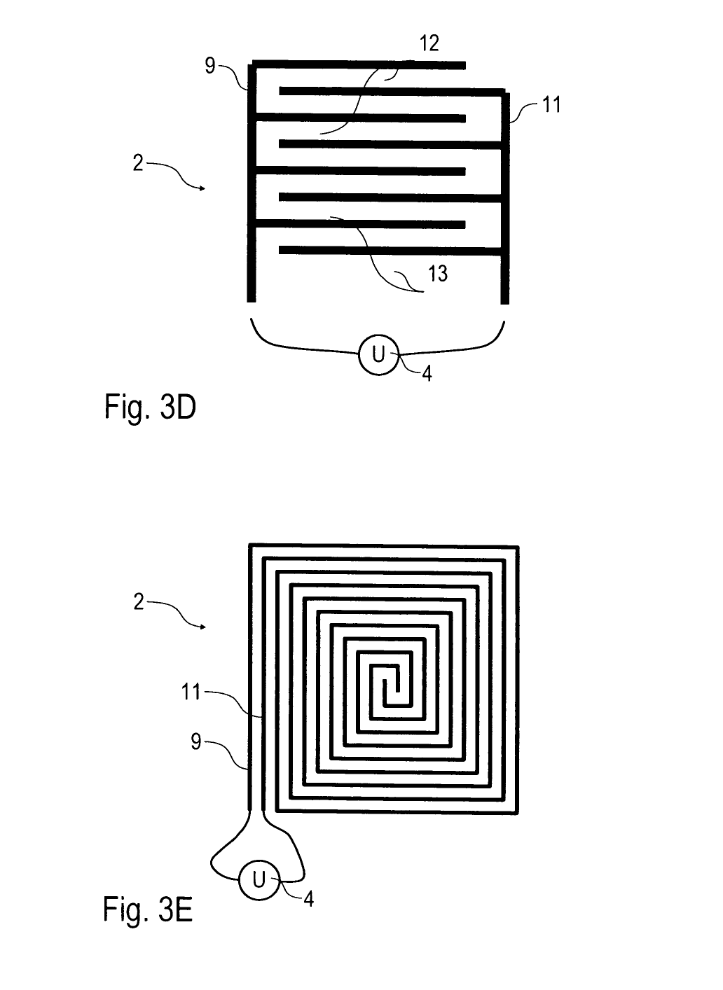

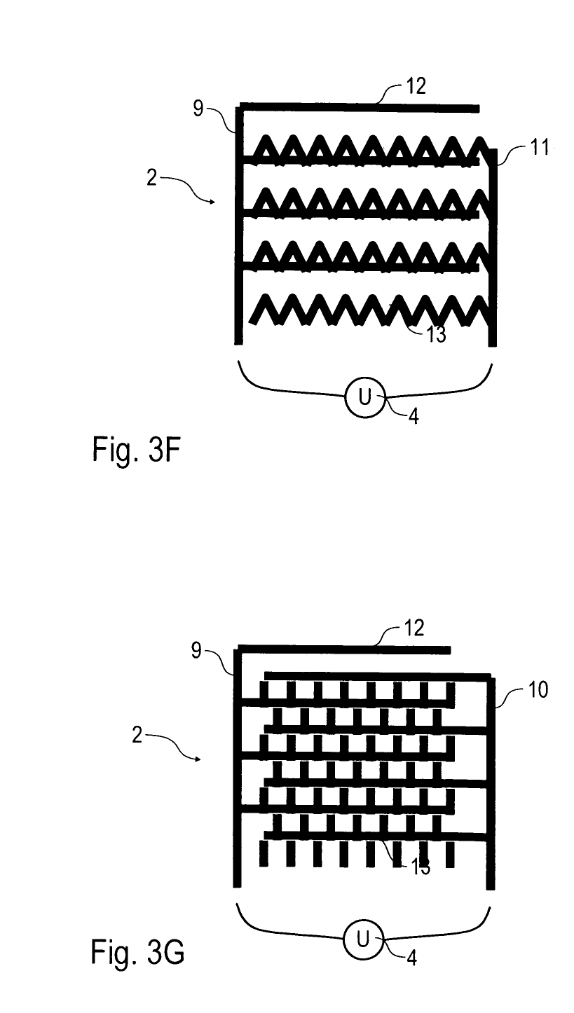

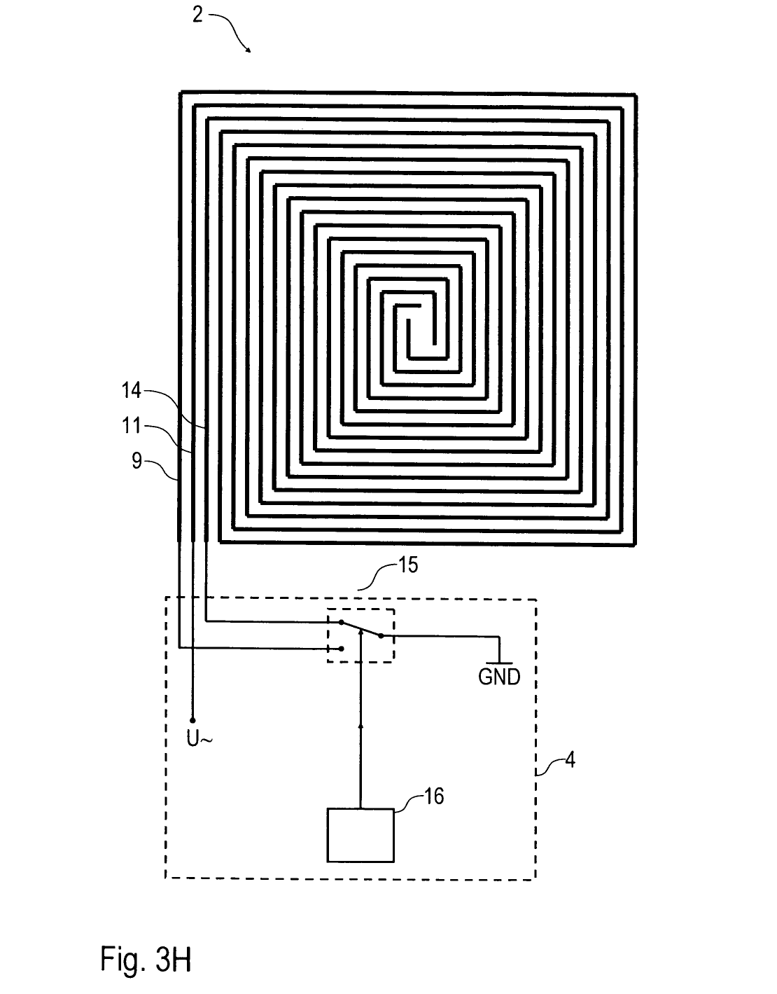

FIG. 3A shows a simplified cross section of a first embodiment of the electrode arrangement 2 of the plasma source 1.

Firstly, it should be noted that the plasma source 1 is integrated into an open cavity of an appliance 6 so that the appliance 6 and the plasma source 1 comprise surfaces 7, 8 which are flush so that the surface 8 of the plasma source 1 constitutes a part of the surface 7 of the appliance 6.

Further, it should be noted that the electrode arrangement 2 of the plasma source comprises several grid-shaped electrodes 9 which are interconnected with each other and embedded into a dielectric layer 10. Further, the electrode arrangement 2 comprises a common back electrode 11 at the under side of the dielectric layer 10.

During operation, the driver circuit 4 applies a AC voltage U to the electrodes 9, 11 so that surface micro-discharges are triggered on the surface 8 of the plasma source 1 as explained in the above-mentioned book titled “Low temperature plasmas”.

FIG. 3B shows a modification of the plasma source 1 according to FIG. 3A, wherein this embodiment corresponds to the previous embodiment to a large extent. Therefore, reference is made to the above description of the embodiment according to FIG. 3A and the same reference numerals are used for corresponding parts and details.

One characteristic of this embodiment is the electrical connection of the upper electrodes 9 which are alternatively connected to the different poles of the driver circuit 4.

FIG. 3C shows another modification of the embodiment of FIG. 3A, wherein this embodiment corresponds to the previous embodiment to a large extent. The difference lies in the mode of operation, the switching of the discharge from one segment to the next. This technique makes use of the plasma afterglow and allows production of large self sterilizing surfaces with low energy requirements.

Therefore, reference is made to the above description with regard to FIG. 3A and the same reference numerals are used for corresponding parts and details.

One characteristic of this embodiment is that the back electrode 11 is missing.

It should further be noted that it is alternatively possible to switch the high voltage lines, as well. Further, it is possible to switch all electrode pairs successively pair by pair.

FIG. 3D shows an alternative design of the electrode arrangement 2, wherein the electrodes 9 and 11 each comprise electrode fingers 12, 13 intertwining each other. In this embodiment, the electrodes 9, 11 along with their electrode fingers 12, 13 are arranged in the same plane.

FIG. 3E shows another design of the electrode arrangement 2 wherein the electrodes 9, 11 are spiral-shaped intertwining each other.

FIG. 3F shows a modification of the electrode arrangement 2 of FIG. 3D, wherein this embodiment corresponds to the embodiment of FIG. 3D to a large extent. Therefore, reference is made to the above description with regard to FIG. 3D and the same reference numerals are used for corresponding parts and details.

One characteristic of this embodiment is that the electrode fingers 13 of the electrode 11 are staggered.

FIG. 3G shows another modification of the electrode arrangement, wherein the electrode fingers 13 of the electrode 11 comprises interlocking branches.

FIG. 3H shows a modification of the electrode arrangement 2 according to FIG. 3E, so that reference is made to the above description and the same reference numerals are used for corresponding parts and details.

One characteristic of this embodiment is that the electrode arrangement 2 comprises an additional electrode 14 besides the electrodes 9, 11. All the electrodes 9, 11, 14 are spiral-shaped intertwining each other.

Further, the driver circuit 4 comprises one switching element 15 connecting the electrodes 9 and 14 alternatively with ground GND, wherein the switching element 15 is controlled by a control device 16.

FIG. 3I shows a modification of the embodiment of FIG. 3H, wherein this embodiment corresponds to the previous embodiment to a large extent. Therefore, reference is made to the above description and the same reference numerals are used for corresponding parts and details.

One characteristic of this embodiment is that the spiral-shaped electrode 11 is staggered.

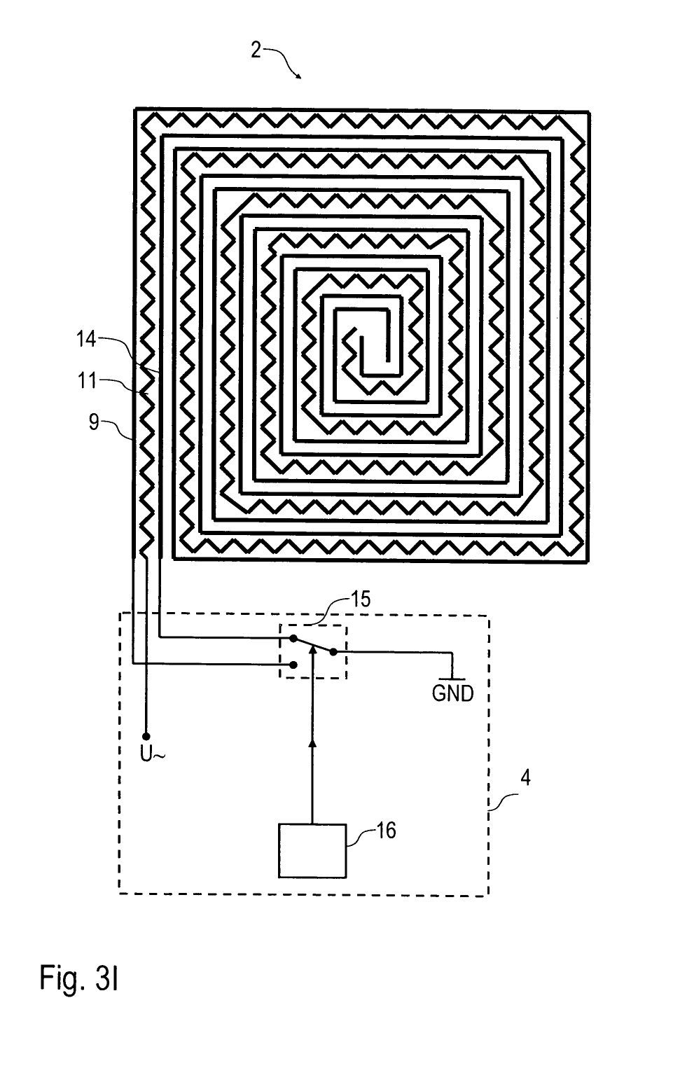

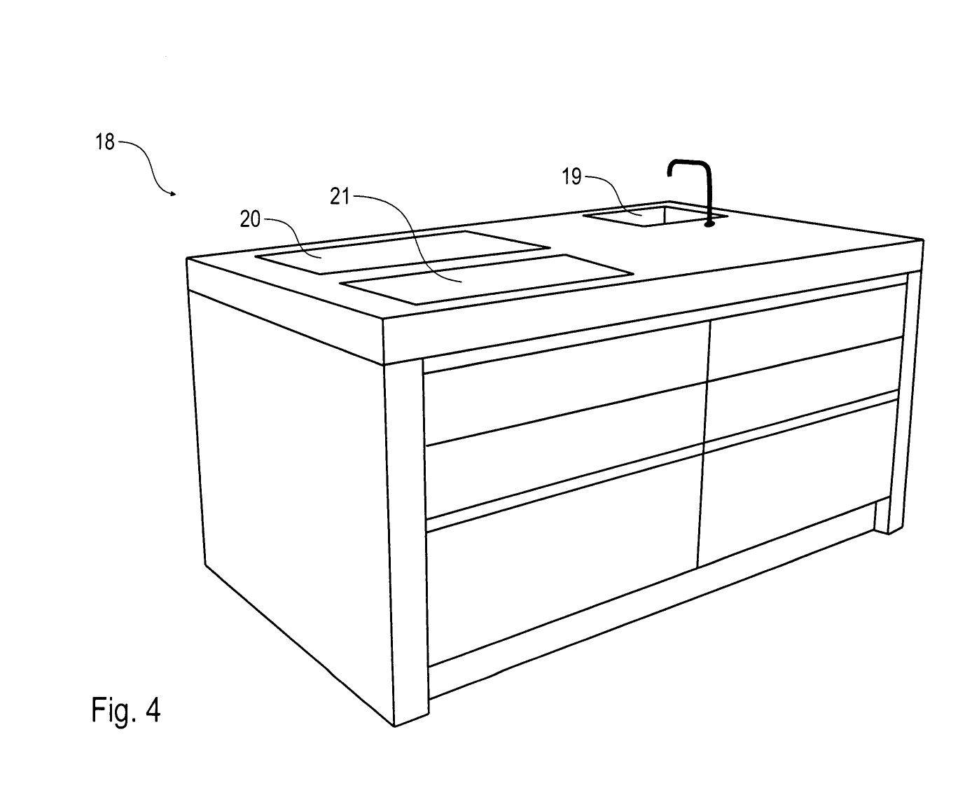

FIG. 4 shows a perspective view of a kitchen block 18 comprising a sink 19 and a ceramic stove top 20 which is per se known from the state of the art. However, the kitchen block 18 additionally comprises a self sterilizing work plate 21 comprising an integrated plasma source as mentioned above. The integrated plasma source generates a low-temperature plasma on the surface of the self-sterilizing work plate 21 thereby sterilizing the surface of the work plate 21 at least partially.

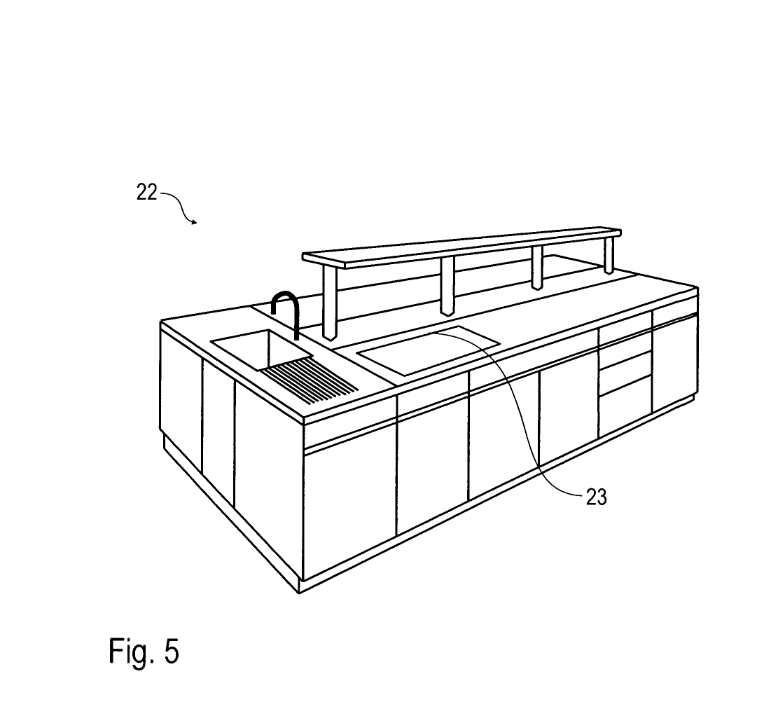

FIG. 5 shows a perspective view of a laboratory table 22 comprising a self-sterilizing work plate 23 similar to the self-sterilizing work plate 21 of the kitchen block 18 according to FIG. 4.

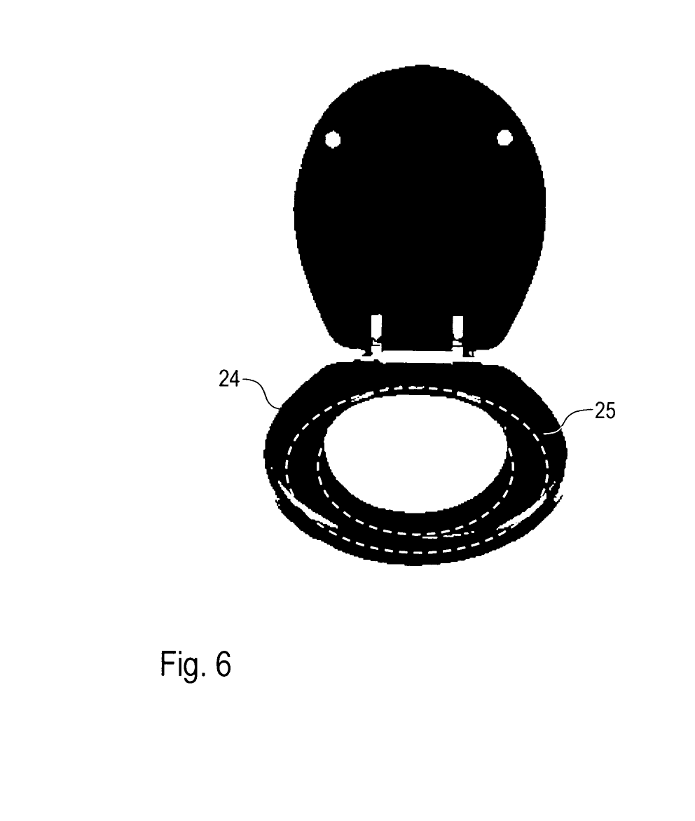

FIG. 6 shows a perspective view of a toilet seat 24 comprising an integrated plasma source 25 which is embedded into the toilet seat 24 and generates a low-temperature plasma on the surface of the toilet seat 24 thereby sterilizing the toilet seat at least partially.

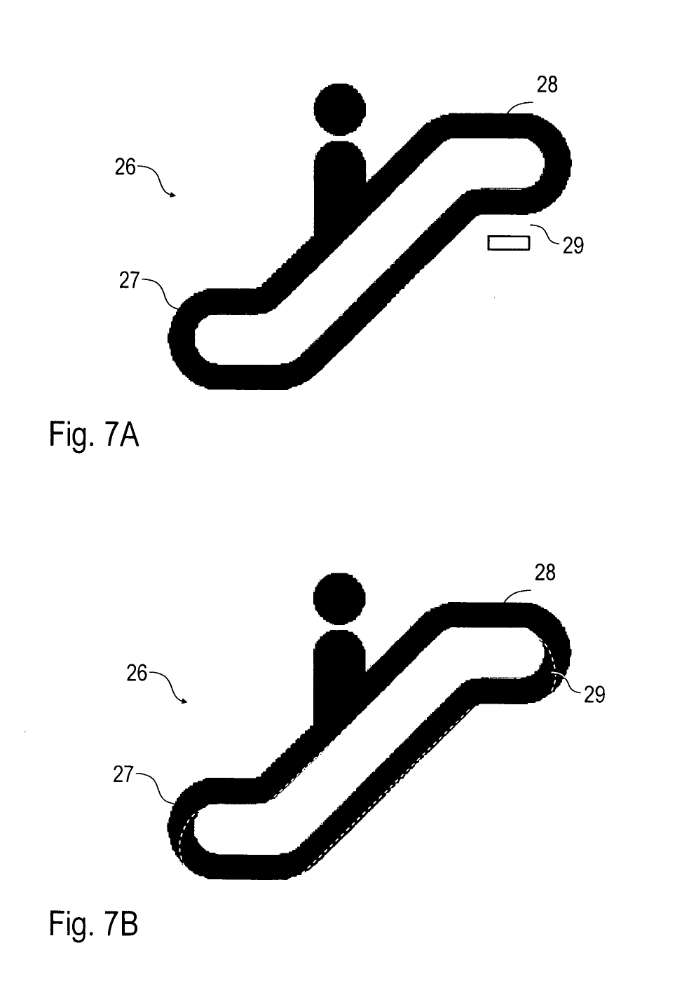

FIG. 7A shows a schematic view of an escalator 26 comprising moving handrails 27, wherein the surface 28 of the moving handrails 27 is sterilized by a stationary plasma source 29 which is arranged beneath the moving handrail 27. The plasma source 29 applies a low-temperature plasma to the surface 28 of the moving handrail 27 thereby sterilizing the surface 28.

FIG. 7B shows a modification of the embodiment of FIG. 7A, wherein this embodiment corresponds to the previous embodiments to a large extent. Therefore, reference is made to the above description and the same reference numerals are used for corresponding parts and details.

One characteristic of this embodiment is that the plasma source 29 is not stationary but arranged within the moving handrail 27 so that the plasma source 29 moves along with the moving handrail 27.

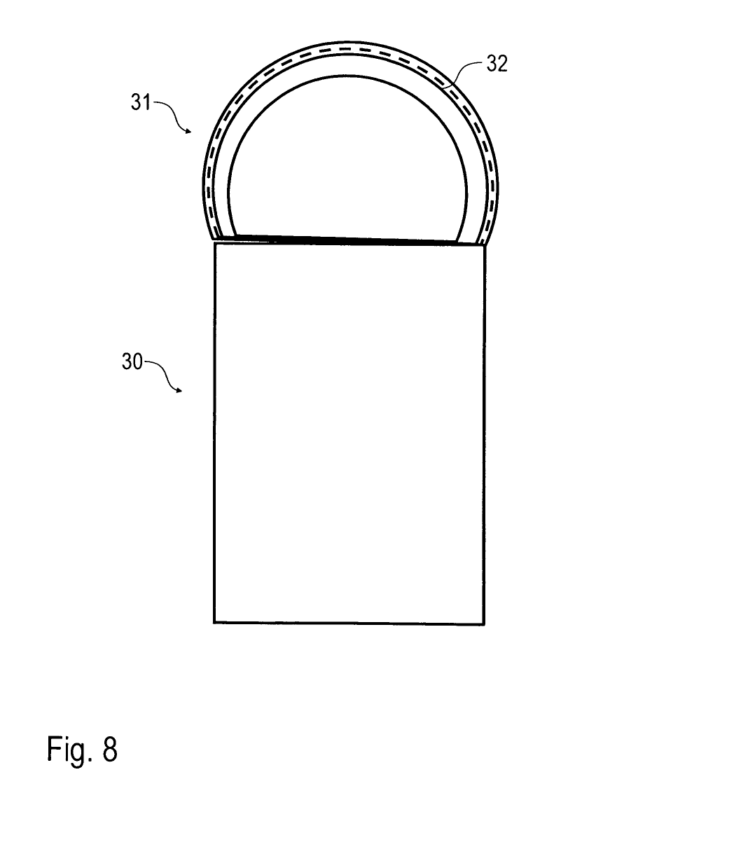

FIG. 8 shows a simplified side view of a deodorant device 30 comprising a ball-shaped head 31 with an integrated plasma source 32, wherein the plasma source 32 generates a low-temperature plasma on the surface of the ball-shaped head 31. The deodorant device 30 is used in the same way as conventional deo-rollers, i.e. the ball-shaped head 31 is moved over the body surface to be sterilized so that the low-temperature plasma generated on the surface of the ball-shaped head 31 sterilizes the body surface.

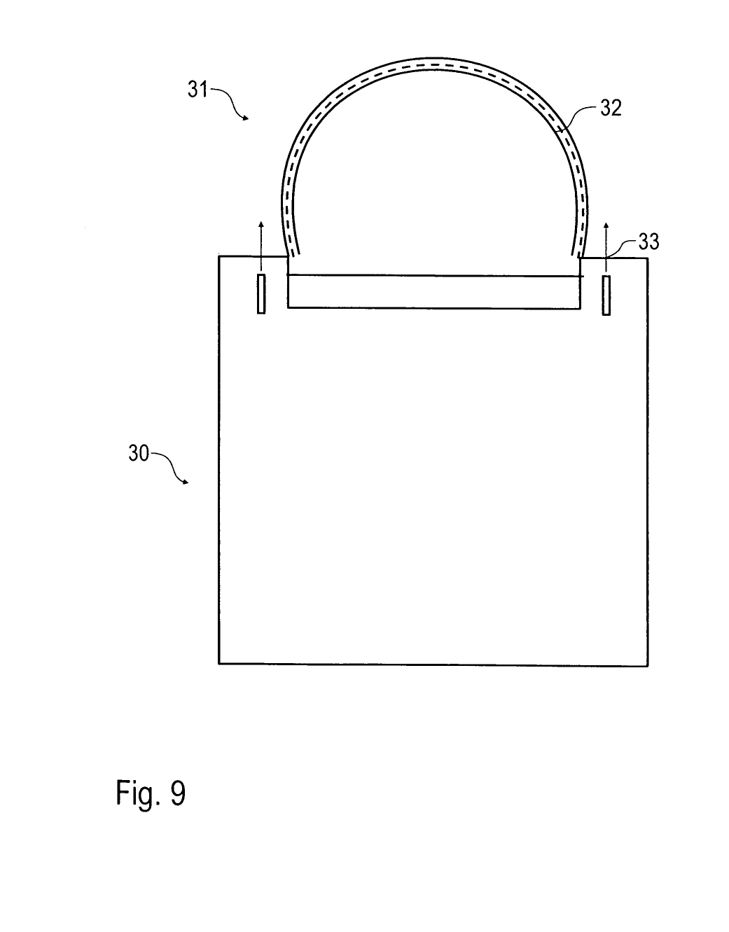

FIG. 9 shows a modification of the deodorant device 30 according to FIG. 8, wherein this embodiment corresponds to the previous embodiment to a large extent. Therefore, reference is made to the above description and the same reference numerals are used for corresponding parts and details.

One characteristic of this embodiment is that the deodorant device 30 additionally comprises nozzles 33 for applying a chemical agent onto the body surface to be sterilized. The chemical agent applied by the nozzles 33 then interacts with the low-temperature plasma thereby enhancing the sterilizing effect of the low-temperature plasma.

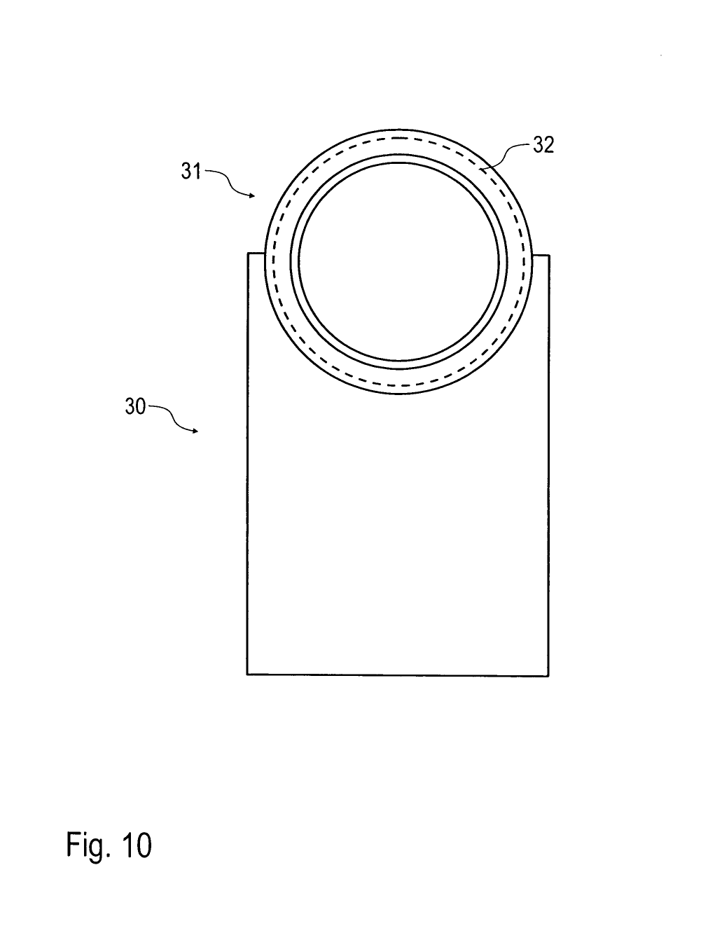

FIG. 10 shows a modification of the deodorant device according to FIG. 8, wherein this embodiment corresponds to the previous embodiment to a large extent. Therefore, reference is made to the above description and the same reference numerals are used for corresponding parts and details.

One characteristic of this embodiment is that the ball-shaped head 31 is in fact rotatable as in conventional deo rollers.

FIG. 11 shows another embodiment of a deodorant device 30 including a plasma source, wherein this embodiment corresponds to the embodiment according to FIG. 8 to a large extend. Therefore, reference is made to the above description and the same reference numerals are used for corresponding parts and details.

The deodorant device 30 depicted in FIG. 11 comprises a housing 51 with a front end 53 being part of the housing 51 of the deodorant device 30. In the embodiment depicted in this figure, the front end has a generally convex shape. Said shape may be varied to a large extend. It is also possible to use a ball shaped front end 53 similar to the embodiment according to FIG. 8 or a flat front end.

The front end 53 has a cylindrical rim which is designed to couple the front end with the rest of the housing 51 which is cylindrical in this case. The housing 51 encloses an interior space of the deodorant device 30 comprising for example among others an electric source for a plasma source of the deodorant device 30.

On top of the front end 53 there is a mesh- or grid-like first outer electrode 57 arranged at the outer surface 59 of the front end 53. The deodorant device 30 comprises a contact ring 61 being electrically connected to the first outer electrode 57 via a conductor 63.

It is clearly to be seen that the first outer electrode 57 is only arranged in the area of the top end of the deodorant device 30. However, it is easily possible to enlarge the first outer electrode 57 in a way that it reaches further down along the outer surface 59 of the front end 53 of the deodorant device 30.

FIG. 12 shows a front view of the deodorant device 30. In this figure the design of the first outer electrode 57 is clearly to be seen. FIG. 12 also shows the conductor 63 which electrically connects the first outer electrode 57 with the contacting ring 61.

The design of the first outer electrode 57 may be amended in a way to adapt the deodorant device 30 to different applications. It is possible to enlarge the first outer electrode 57 to generate more plasma if necessary. It is also possible to amend the design of the first outer electrode 57 to be applicable also to a delicate and sensitive skin of a user of the deodorant device 30.

The front view of the first outer electrode 57 shown in FIG. 12 shows that the electrode comprises three eccentric circles, the centre of which is arranged at the centre of the front end 53. Between the concentric circles regularly arranged parts of the electrode are connecting two or more of the concentric rings. It is clearly to be seen that also neander-shaped lines of conductive material may be used to realize the first outer electrode 57. Additionally, comb-like lines may be arranged on the outer surface 59 of the front end 53 to realize the first outer electrode 57.

The material used for realizing the first outer electrode preferably is chosen to be inert and rust-proof especially against moisture and aggressive fluid.

In FIG. 13 there is a longitudinal cut of the enlarged front end 53 of the deodorant device 30. It is clearly to be seen that the front end is convex. The embodiment shown in FIGS. 11 to 13 has a ball-shaped top end. That is why the upper end of the front end 53 is curved like a spherical segment. In the embodiment the spherical segment is arranged on a conical segment.

The front end 53 is hollow. Its rim 55 is preferably connected to the rest of the housing 51 via a snap-on connection. It is also possible to glue the front end 53 to the rest of the housing 51 to make sure that the deodorant device 30 is moisture-proof closed to protect the plasma source with its circuits and the energy source within the interior space 65 of the housing 51. In case the deodorant device 30 is designed as a disposable the complete housing 51 may be closed moisture-proof.

To enable a user to replace the energy source, at least one end of the housing 51 should be closed by a removable cap. If the front end 53 is glued to the rest of the housing 51, it is possible to provide a removable lid, for example at the opposite end of the deodorant device 30.

In the embodiment of the invention, the front end 53 is designed as a removable cap, to allow an exchange of the front end 53 in case the first outer electrode 57 may be worn or destroyed or in case a user would like to use different electrodes because of a delicate and sensitive skin.

In the embodiment shown in FIGS. 11 to 13, the front end 53 is designed as a hollow cap including a free space 67 and having an inner surface 69 as well as an outer surface 59, carrying the first outer electrode 57. On the inner surface 69 a second inner electrode 71 is provided. It comprises or is made of a conductive material. In a preferred embodiment the inner surface 69 is completely coated with a conductive substance, for example metal. It is also preferred to use a conducting glue placed on the inner surface 69 of the front end 53, which comprises or preferably is made of non-conductive material, especially of plastics, more especially of Teflon because this kind of plastics is characterized by very smooth gliding properties; additionally it only very little irritates the skin of a user. The main aspect of the body of the front end 53 is to be non-conductive. That is why also glass, ceramics or other insulating materials can be used to realize the front end 53 of the deodorant device 30.

In a preferred embodiment of the deodorant device 30 the front end 53 with the first outer electrode 57 and the second inner electrode 71 are produced by injection moulding. In this case different plastic materials are used. Conductive plastic material is used to realize the electrodes 57 and 71, while non-conductive material is used for the body of the front end 53. Using this method to produce the front end 53, it is very easy to realize different shapes for the second inner electrode 71 and especially for the first outer electrode 57. In most cases the second inner electrode 71 covers the whole interior surface 69 of the cap-like front end 53. The first outer electrode 57 may be realized as a grid or a mesh, wherein it is easily possible to realize different shapes of grids and/or meshes to adapt the first outer electrode 57 to different applications. In some cases users may prefer a deodorant device 30 generating more plasma. In other cases the user, having a sensitive and delicate skin, may prefer a deodorant device 30 producing less plasma to not irritate the skin. That is why users may prefer larger or smaller first outer electrodes 53 on the outer surface 59 of the front end 53.

Different designs and dimensions, especially of the first outer electrode 57 may not only be realized by using said injection moulding but also when producing grid- or mesh-like first outer electrodes 57 with other methods mentioned above.

In another preferred embodiment, the first outer electrode 57 may be realized by using thin wires, which are arranged grid- or mesh-like and which are then embedded within plastic material, which will be brought onto the wire preferably using an injection moulding process. A cup-like second inner electrode 71 may then be placed onto the inner side of the front end 53. However, the second inner electrode 71 may also be realized by depositing conductive material like metal or glue on the interior surface of the front end 53.

The first outer electrode 57 comprises or preferably is made of a conductive material. Preferably the outer surface 59 is provided with grooves showing a pattern as it is to be seen in FIG. 12. The grooves are filled with a conductive material. It is possible to deposit a conducting substance within the grooves, for example metal or to fill the grooves with conducting glue or another conducting substance which will stick in the grooves. Also the conductor 63 is made the same way.

In another preferred embodiment the whole outer surface 59 of the front end 53 is coated with a conductive substance. Afterwards the conductive substance is removed from the outer surface 59 and only the grid-like pattern of the first outer electrode 57 and the conductor 63 will remain on the outer surface 59 of the front end 53.

The first outer electrode 57 gets in contact with the contacting ring 61 via the conductor 63. The contact ring 61 is electrically connected to the plasma source 75 arranged within the housing 51. FIG. 13 shows that the contact ring 61 is connected via a contact pin 73 which is electrically connected to the contact ring 61. Preferably the contact pin 73 is pressed against the contact ring 61 by an elastic element. It is easily to be seen that it is only important to achieve an electrical connection between the plasma source 75 and the conductor 63. That is why a little contacting element instead of a complete ring between the contact pin 73 would suffice.

The second inner electrode 71 is connected via a connector, not depicted in FIG. 13, to a connecting plate 77 of the plasma source 75.

From the explanation above it is to be seen that the plasma source 75 comprises a first outer electrode 57 being electrically connected via the connector 63, the contact ring 61 and the contact pin 73 to the plasma source 75. The plasma source also comprises a second inner electrode 71 which is conductively connected to the connecting plate 77 of the plasma source 75. The plasma source 75 is supplied by an electric source 79 arranged in the interior space 65 of the housing 51. The electric source 79 may comprise one or more batteries or accumulators to energize the plasma source 75. The housing encloses additional circuitry, one or more switches and so on to activate the deodorant device 30 if needed. Preferably the output of the plasma source 75 may be adjustable by a user.

Also the embodiment depicted in FIGS. 11 to 13 of the deodorant device 30 may comprise nozzles for applying a chemical agent onto the body surface of a user. Such nozzles are not shown in FIGS. 11 to 13, but for example in FIG. 9.

From the description related to FIGS. 11 to 13 it is easily to be seen that the deodorant device 30 may be realized without using any movable parts. Additionally, it is possible to exchange the front end 53 of the housing 51 to replace damaged electrodes or to use different electrodes for more or less sensitive skins or when using different chemical agents together with the deodorant device 30.

Referring to FIGS. 11 to 13 and to the description of the configuration of the deodorant device 30 depicted in said figures, it is clearly to be seen that using the deodorant device 30, plasma will be generated at the front side of the housing 51, i.e. in the area of the first outer electrode 57 of the front end 53. A user may move the deodorant device 30 at a distance to his skin or directly touching the skin with the front end 53 of the deodorant device 30. It is also possible to move the deodorant device 30 at a distance to a shirt of a user. The plasma emanating from the first outer electrode 57 will penetrate the fabric of the cloth of the shirt and will reach the skin of the user if the distance is not too large. Additionally, it is possible to touch the outer surface of the shirt with the front end 53 of the deodorant device 30. In this case the plasma will mainly be generated at the far end of the first outer electrode 57, i.e. within the shirt of the user. It is possible that also in this case, when touching the fabric of a shirt, additionally plasma will be generated a the surface of the first outside electrode 57 and within the fabric of the shirt. This will result in a decontamination and disinfection of the outer surface of the shirt and the shirt itself, while the plasma generated at the far end of the fabric of the shirt will decontaminate and disinfect the skin of the user.

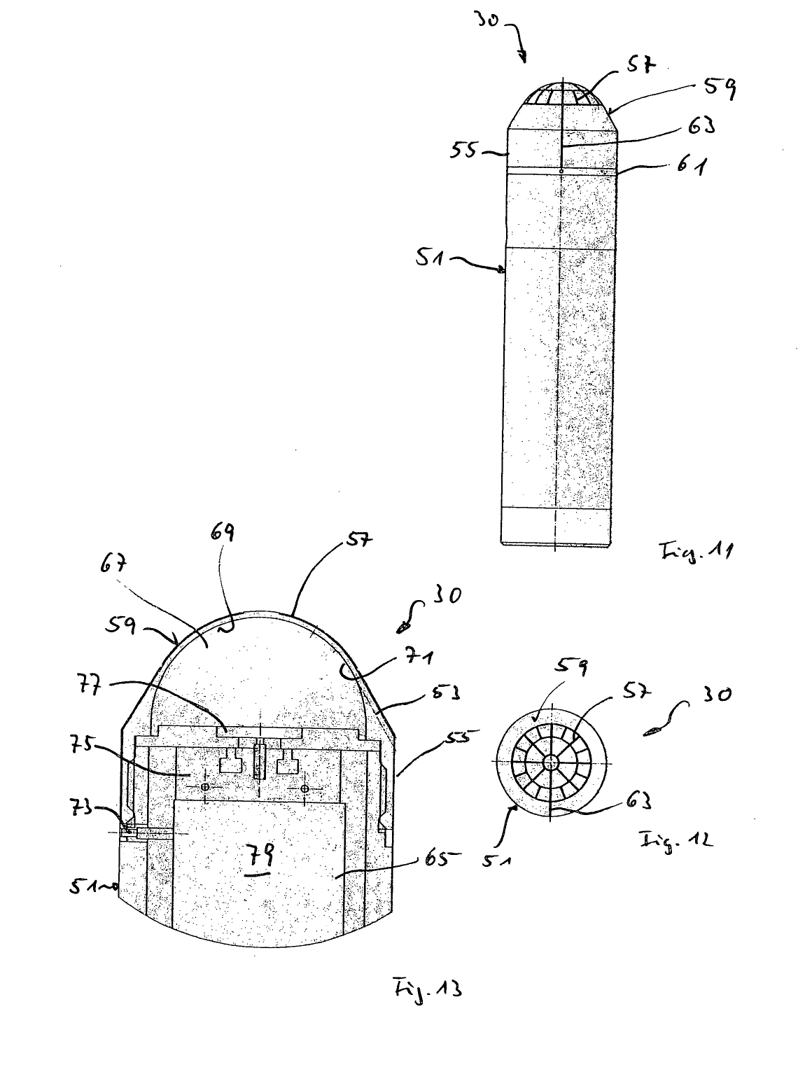

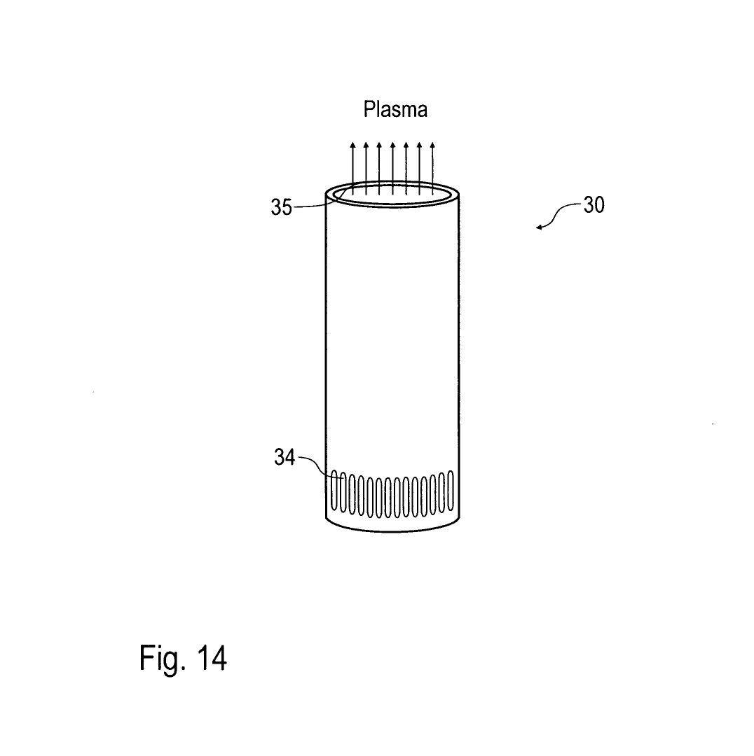

FIG. 14 shows another embodiment of a deodorant device 30 including a plasma source in the form of a so-called plasma jet which draws in ambient air through inlet openings 34 at the bottom of the deodorant device 30, while the low-temperature plasma is applied through an outlet opening 35 at the top of the deodorant device 30.

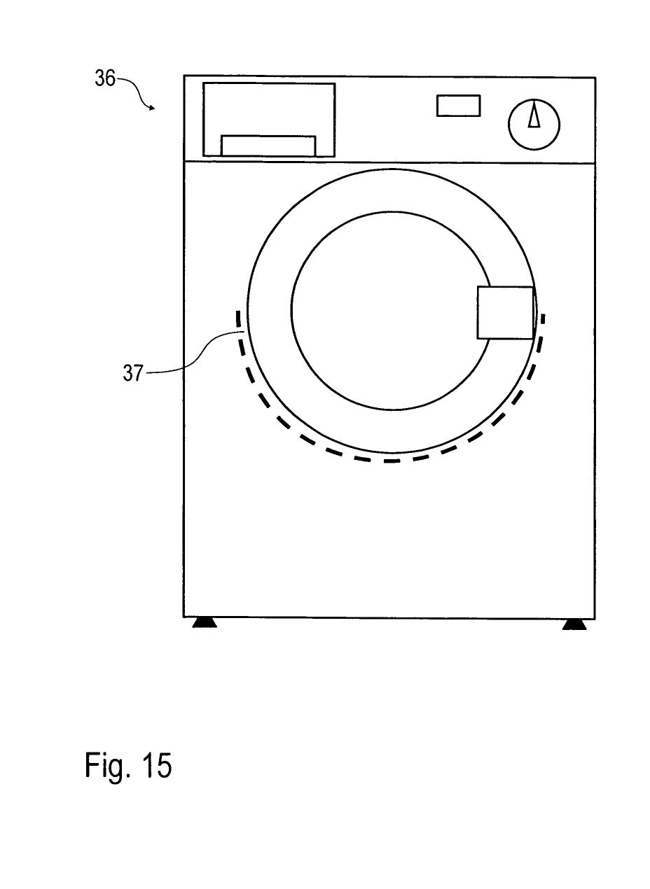

FIG. 15 shows a schematic view of a washing machine 36 including a plasma source 37 for applying a non-thermal plasma to the clothes within the washing machine 36.

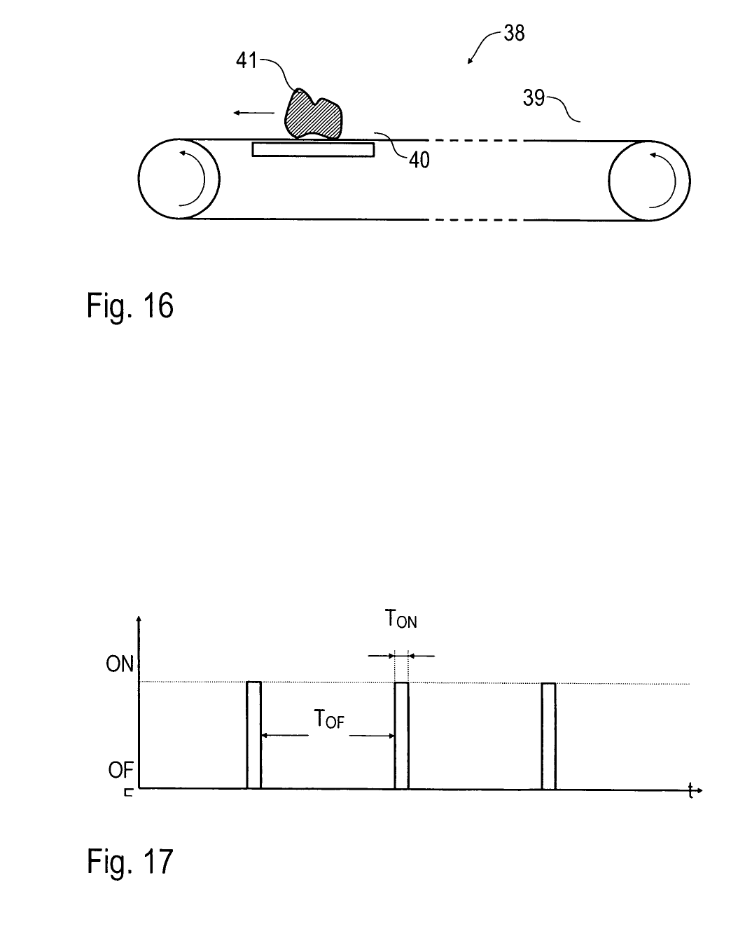

Moreover, FIG. 16 shows a schematic side view of a conveyor 38 comprising a conveyor belt 39 and a plasma source 40 which is arranged beneath the upper conveyor belt 39. The plasma source 40 applies a non-thermal plasma through the permeable upper conveyor belt 39 to objects 41 thereby sterilizing the upper surface of the upper conveyor belt 39 and/or the objects 41 on the conveyor belt 39.

FIG. 17 shows a diagram illustrating the on- and off-times of the plasma sources mentioned above. Preferably, the off-time T_{OFF} is much longer than the on-time T_{ON}. In this connection, it should be noted that the sterilization/disinfection also occurs during the off-time T_{OFF} due to the so-called after-glow effect.

FIG. 18 shows another embodiment wherein the surface to be disinfected and/or sterilized and/or decontaminated is separated from the appliance including the plasma source. In this case, the invention encompasses a container 81 including one or more objects or a substance to be exposed to plasma.

The container 81 comprising a first element, preferably a body enclosing an interior space 85 wherein the objects or substances are placed. In the embodiment depicted in FIG. 8 the container 81 is filled with a substance 87, for example foodstuff or the like.

The container 81 comprises a lid 89 which is placed at the opening of the body 83 to tightly close the container 81. The connection between the lid 89 and the body 83 may be realized by gluing, melting, ultrasonic welding or another known method.

The lid 89 comprises a first electrode which will be explained in relation to the following figures.

FIG. 18 shows that a counter electrode 91 is placed onto the lid 89 and comprises connecting cables 93 connecting the counter electrode 91 with an electric source 95 powering the electrodes to generate plasma 97, indicated by arrows, within the container 81. Preferably cold atmospheric plasma will be generated by the electrodes mentioned above.

The counter electrode 91 preferably is realized as a stamp-like element, especially as a stamp, the counter electrode 91 itself being the basic element of the stamp and having a handle H which is made, as the counter electrode 91 itself, of insulating material. The connecting cables 93 are embedded in the handle H.

FIG. 19 shows a top view onto the lid 89. It comprises a first electrode 101, comprising strip-like conductive areas, for example comb-like arranged wires 103 connected to a contact 105 which is meant to realize a connection to ground.

Preferably, the wire 103 is very thin having a diameter of about 5 μm to 18 μm, preferably of 10 μm to 50 μm.

The distance between two comb-like arranged wires is chosen in a range between 1 mm to 10 mm, preferably between 2 mm to 5 mm.

The wire 103 preferably is embedded within the body of the lid 89 to be protected against damages. However, it is also preferred to realize the first electrode 101 by depositing a conductive material, preferably metal, on the inner surface of the lid 89. The lid itself is made of insulating material, preferably of plastics. In this case preferably the wire 103 can be embedded within the lid, but it is also preferred to deposit a conductive material on the inner surface of the lid 89. It is also preferred to totally cover the inner surface of the lid 89 with a conductive substance and take off all the substance from the inner surface except from the area where the inner electrode is realized.

The contact 105 may be realized the same way, namely by embedding a conductive element, preferably made of metal, within the lid and connecting the wire to it.

The first electrode probably is realized by injection moulding the lid using two components. The main part of the lid 89 is made of non-conducive plastic while the first electrode 103 and the contact 105 is made of conductive plastic. It is also preferred to realize notches within the inner surface of the lid 89 having the shape of the inner electrode 101, i.e. being arranged in a comb-like pattern, and to fill the notches with conductive material, for example conductive glue or to deposit a conductive substance within the notches. Also the contact 105 can be realized this way, i.e. by preparing a cavity in the inner surface and to fill it with conductive material.

The pattern of the inner electrode may be varied. For example it is possible to realize two comb-like areas, engaging each other without getting in contact. Also meandric or wave-like first electrodes 101 can be realized in this case.

FIG. 19 shows an outer edge e of the lid 89 and a hachure h indicating the area, where the lid 89 is tightly connected to the body 83 of the container 81.

FIG. 20 shows an enlarged cross section through the lid 89. The insulting basic material 107, the lid 89 preferably is made of, and the wire 103 are clearly to be seen, also the contact 105, being in embedded in the inner surface 109 of the lid 89.

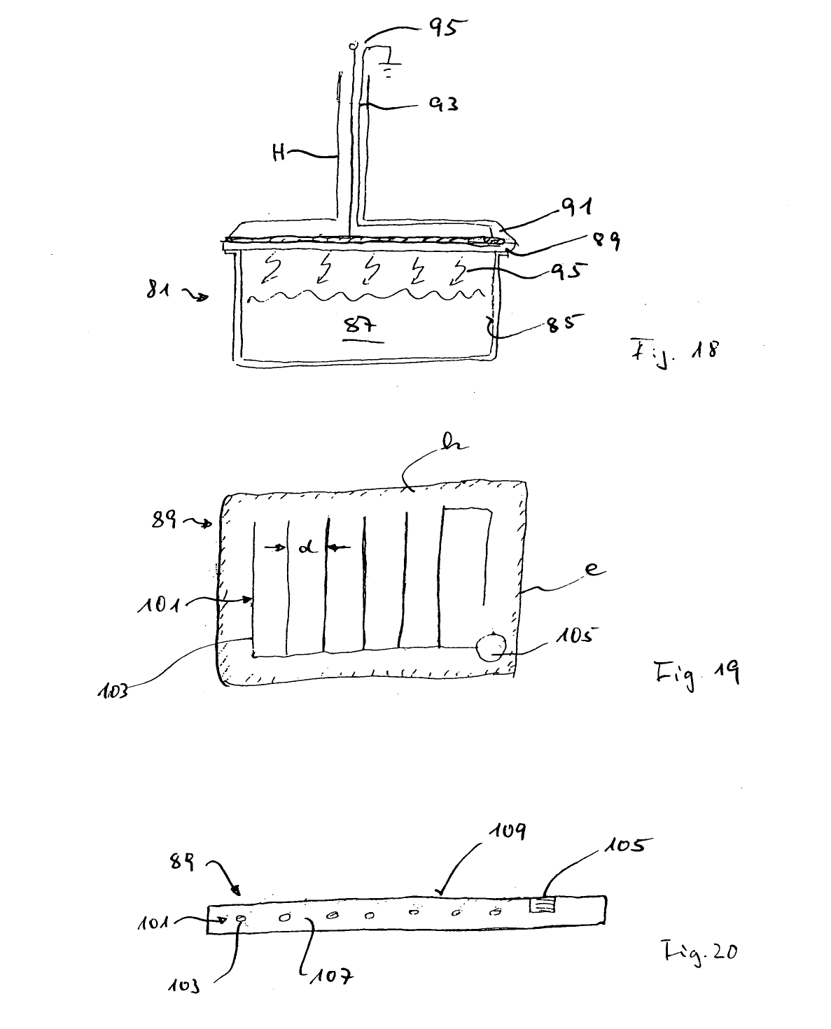

FIG. 21 shows a top view of the counter electrode 91. It is clearly to be seen that the lower surface of said electrode, which will be placed on the lid 81, comprises a second electrode, realized as an conductive area 111 comprising or being made of conducting material. In the embodiment of the counter electrode 91 depicted in FIG. 21 the area 111 is made of a metal layer, preferably of stainless steel. The surface of the counter electrode 91 shown in FIG. 21 also comprises a contact area 113 not being electrically connected to the area 111 and being arranged at an area to be electrically connected to the contact 105 in case the counter electrode 91 is placed on the lid 89, as shown in FIG. 18. The area 111 and the contact area 113 of the counter electrode 91 are connected to the connecting cables 93 and to the electric source 95 shown in FIG. 18.

To activate the plasma 97 within the container 81, the stamp-like counter electrode 91 will be placed onto the lid 89 and the electric source 95 will be activated. Powering the electrodes, the counter electrode 91, i.e. the conductive area 111, plasma 97 will be generated at the first electrode 101 of the lid 89. The plasma disinfects and/or sterilizes and/or decontaminates the content of the container 81, i.e. the free area under the lid 89 and the content, for example the foodstuff 87.

The lid 89 described in relation to FIGS. 18 to 20 is preferably made of a more or less rigid material. The counter electrode 91, also being made of rigid material may easily be brought in contact with the lid 89 using a sufficient pressure to activate the first electrode 101 of the lid 89 when activating the second electrode, i.e. the area 111, of the counter electrode 91 by the electric source 95.

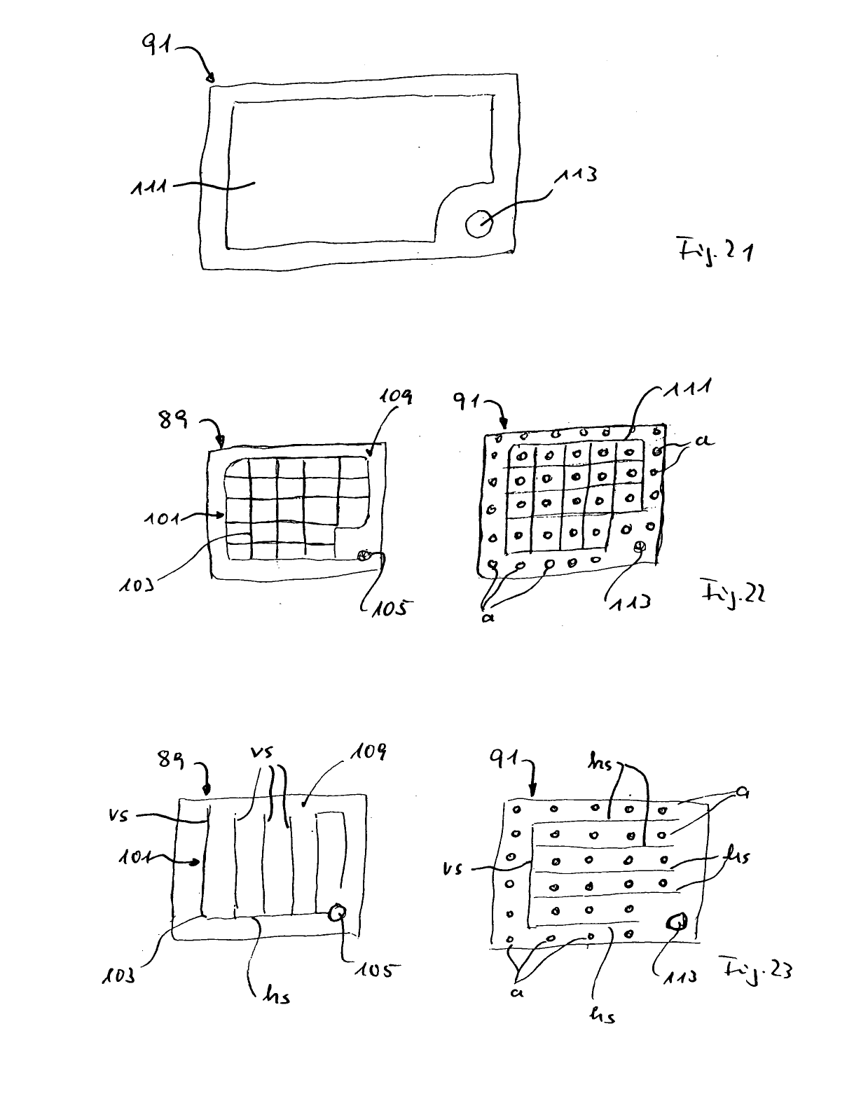

FIG. 22 shows a lid and a counter electrode which are used in case the lid 89 and possibly the container 81 are soft and bendable. Both elements correspond to the embodiment according to FIGS. 18 to 21 to a large extent. Therefore reference is made to the above description, and the same reference numerals are used for corresponding parts and details.

At the left side of FIG. 22 the lid 89 is depicted in bottom view showing the inner surface 109 of the lid 89. At the right side of FIG. 22 the counter electrode 91 is also depicted in bottom view. In case the lid 89 is made of soft and bendable material, the structure of the first electrode 101 and the structure of the area 111, comprising or made of conducting material, are very similar, preferably more or less identical. For example, the first electrode 101 comprises wires 103 running vertically to each other, i.e. a number of wires is arranged horizontally in FIG. 22 and a number of wires is arranged vertically. Accordingly, the conductive area 111 of the counter electrode 91 is realized by vertically and horizontally arranged lines of conductive material. The pattern of the wire of the lid 89 and the pattern of the conductive area 111 of the counter electrode 91 is as similar as possible.

FIG. 22 shows that the counter electrode 91 comprises a number apertures a being connected to suction means applying a negative pressure to the surface of the counter electrode. In the embodiment depicted in FIG. 22, the counter electrode 91 comprises a pattern of apertures a being arranged in vertical or horizontal lines. Preferably, each square between horizontal and vertical lines of conductive material of the conductive area 111 comprises at least aperture a.

Preferably, also in this embodiment, the counter electrode 91 is realized like a stamp. In case the stamp is lowered to the soft and bendable lid 89 to activate the plasma 95 a suction means is activated to suck the lid 89 against the surface of the counter electrode 91. By this the electrodes of the lid 89 and the counter electrode 91 are arranged in a defined position, preferably parallel to each other. The contact 105 of the lid 89 and the contact 113 of the counter electrode 91 are pressed together to realize a conductive contact between both contacts. Energizing the first electrode 101 and the second electrode, i.e. the conducting area 111 of the counter electrode 91 will result in a generation of plasma 95.

FIG. 23 shows another embodiment of a lid (left-hand) and a counter electrode (right-hand). This embodiment corresponds to the embodiment according to FIG. 22 to a large extent. Therefore reference is made to the above description and the same reference numerals are used for corresponding parts and details.

It is clearly to be seen that the lid 89 and the counter electrode 91 only differ from the embodiment depicted in FIG. 22 by having different electrodes.

The first electrode 101 at the inner surface 109 of the lid 89 is realized as depicted and described in FIG. 19 and comprises strip-like conductive areas, especially realized by a wire 103. That is why reference is made to the description of said Figure. The wire 103 of the first electrode 101 is arranged comb-like having a number of vertical sections vs of wire, said wires being connected to each other by a horizontal section hs of a wire which is connected to the contact 105.

The second electrode, i.e. the conductive area 111 of the counter electrode 91 is realized by a number of horizontal, strip-like areas hs of conducting material, especially wires, being connected by a vertical section vs of a wire or connecting area. Also in this case a comb-like pattern is realized.

From FIG. 23 it is easily to be taken that the comb-like pattern of the first electrode 101 of the lid 89 comprises a number of vertically oriented sections, while the comb-like pattern of the second electrode, i.e. conductive area 111 of the counter electrode 91, comprises a number of horizontally oriented sections. In other words, the conductive strip-like areas of the first electrode 101 realize a first pattern, wherein the conductive sections are oriented in a first (vertical) direction, while the conductive strip-like areas of the second electrode, i.e. conductive area 111, of the counter electrode 91, realize a second pattern, wherein the conductive sections are oriented in a second (horizontal) direction. The strip-like areas of the first and second electrodes include an angle a of preferably 90°. This embodiment is shown in FIG. 23. The angle between said strip-like areas may be chosen in a range of 0°<α<180°, preferably of 45°≦135°.

Also in this embodiment, the counter electrode 91 comprises apertures a as mentioned above and a contact area 113, as described in relation to FIG. 21.

To generate plasma 95 within the container 81 the—preferably stamp-like—counter electrode 91 is lowered onto the upper surface of the lid 89 and a pump or the like is activated to suck air through the apertures a. This will result in sucking the soft and bendable lid 89 against the counter electrode 91.

At the crossing areas of the first electrode 101 and the conductive area 111 of the lid and the counter electrode 91, plasma will be generated if the electric source 95 is activated.

The embodiment shown in FIG. 23 will allow to generate plasma 95 in case it is not possible to realize very similar electrodes (first electrode 101 of the lid 89 and conducting area 111 of the counter electrode 91) as shown and described in FIG. 22.

It is clearly to be seen that it is possible to generate plasma within a container 81 comprising a mostly solid and stable lid 89 as described in relation to FIGS. 18 to 21. However, it is also possible to generate plasma 95 within a container 81 which is tightly closed by a soft and bendable lid as described in relation to FIGS. 22 and 23.

When filling a container 81 with objects or a substance, especially foodstuff 87 or medical substances, preferably first of all the container 81 will be disinfected and/or sterilized and/or decontaminated, especially with plasma, before filling it. After filling said container 81 the lid 89 will be firmly, especially tightly connected to the body 83 of the container 81. Then the counter electrode 91 will be placed onto the lid 89. In case it is a stable lid, the first electrode 101 of the lid 89 and the second electrode, i.e. the conducting area 111 of the counter electrode 91 are arranged closely to each other in a defined distance.

In case of soft and bendable lids 89, as described in relation to FIGS. 22 and 23, the electrodes may not be arranged close enough to each other to generate plasma after activating the electric source 95. That is why it will be necessary in this case, i.e. using soft and bendable lids, to activate a pump or other mean to suck air through the apertures a of the counter electrode 91 and to suck out the air between the counter electrode 91 and the lid 89. By this the electrode 101 of the lid 89 and the conducting area 111 of the counter electrode 91 will be arranged closely to each other. It is now possible to generate plasma within the container 81.

Generally, it is only necessary to activate the power source 95, for example for 2 to 10 seconds. Even after switching off the power source 95 there will be an after glow within the container 81 resulting in a continued disinfection and/or sterilization and/or decontamination of the interior space 109 of the container 81 and its content.

It is clearly to be seen that the basic idea of disinfecting, sterilizing and/or decontaminating an appliance may easily be used in generating plasma within a container 81.

Although the invention has been described with reference to the particular arrangement of parts, features and the like, these are not intended to exhaust all possible arrangements of features, and indeed many other modifications and variations will be ascertainable to those of skill in the art.

### LIST OF REFERENCE NUMERALS

1 Plasma source
2 Electrode arrangement
3 Housing
4 Driver circuit (high voltage power supply)
5 Connection cable
6 Appliance
7 Surface of the appliance
8 Surface of the plasma source
9 Electrodes
10 Dielectric layer
11 Back electrode
12 Electrode finger
13 Electrode finger
14 Electrode
15 Switching element
16 Control device
18 Kitchen block
19 Sink
20 Ceramic stove top
21 Workplate
22 Laboratory table
23 Workplate
24 Toilet seat
25 Plasma source
26 Escalator
27 Moving handrails
28 Surface of the handrails
29 Plasma source
30 Deodorant device
31 Ball-shaped head
32 Plasma source
33 Nozzles
34 Inlet openings
35 Outlet opening
36 Washing machine
37 Plasma source
38 Conveyor
39 Conveyor belt
40 Plasma source
41 Objects
GND Ground
51 Housing
53 Front end
55 Rim
57 First outer electrode
59 Outer surface
61 Contact ring
63 Conductor
65 Interior space
67 Free space
69 Inner surface
71 Second inner electrode
73 Contact pin
75 Plasma source
77 Connecting plate
79 Electric source
81 Container
83 Body
85 Interior space
87 Foodstuff
89 Lid
91 Counter electrode
93 Connecting cables
95 Electric source
97 Plasma
101 First electrode
103 Wire
105 Contact
107 Basic material
109 Inner surface
111 Area
113 Contact area
H Handle
a Apertures
d Distance
e Outer edge
h Hachure
vs Vertical sections
hs Horizontal sections

1. An appliance for at least partially sterilizing a contaminated surface comprising an integrated plasma source for at least partially sterilizing the surface by generating a non-thermal plasma on the surface thereby reducing a concentration of pathogenic germs on the surface.

2. The appliance according to claim 1, wherein the surface to be sterilized is a surface of the appliance which is contaminated during use of the appliance.

3. The appliance according to claim 1, wherein
a) the plasma source is a surface micro-discharge plasma source comprising several electrodes, and/or
b) there is a uniform distance between adjacent electrodes of different polarity, or
c) there is a spatially variable distance between the adjacent electrodes of different polarity.

4. The appliance according to claim 3, wherein the surface micro-discharge plasma source is embedded into the surface of the appliance, so that the non-thermal plasma is generated on top of the surface of the appliance.

5. The appliance according to claim 4, wherein
a) the electrodes of the surface micro-discharge plasma source have a shape which resembles the shape of the surface of the appliance, and/or
b) the electrodes of the surface micro-discharge plasma source are flat and/or planar.

6. The appliance according to claim 3, wherein the electrodes of the surface micro-discharge plasma source are arranged in the same plane.

7. The appliance according to claim 6, wherein
a) the electrodes are finger-shaped intertwining each other from opposite directions, or
b) the electrodes are spiral-shaped intertwining each other, and/or
c) the electrodes comprise interlocking branches or kinks.

8. The appliance according to claim 3, wherein
a) electrically opposite electrodes of the surface micro-discharge plasma source are arranged in separate adjacent electrode layers, and/or
b) each of the electrode layers is planar and the separate electrode layers are arranged coplanar relative to each other, and/or
c) the electrodes are switched at a constant or variable frequency.

9. The appliance according to claim 1, wherein
a) the surface to be sterilized is substantially planar, and/or
b) the plasma source comprises a substantially planar surface which is flush with the surface which is to be sterilized.

10. The appliance according to claim 1, wherein the appliance is
a) water-proof,
b) dust-proof,
c) air-borne particles proof, and/or
d) easy to clean.

11. The appliance according to claim 3, wherein the surface consists of a corrosion resistant material.

12. The appliance according to claim 1, wherein the appliance is
a) a work plate on a kitchen table or on a laboratory table, or
b) a cutting board for cutting objects, or
c) a handle, or
d) bathroom equipment, or
e) a deodorant device for deodorizing a body surface, or
f) a moving handrail of an escalator or a moving walkway, or
g) gym equipment, or
h) a rechargeable device for mobile sterilization of surfaces, or
i) a battery operated device for reducing itching caused by insect bites, or
j) suitable for protection against athletes' foot and other fungal diseases, or
k) suitable for disinfection of baby bottles, pacifiers, toys, dentures, tooth brushes, hair brushes, or
l) a dishwasher or a dryer or
m) a conveyor comprising a conveyor belt, wherein the plasma source is arranged in a vicinity of the conveyor belt so that the plasma sterilizes objects which are conveyed on the conveyor belt, or
n) a washing machine, or
o) a device for disinfecting hands, or
p) a container, or
q) a kitchen appliance, or
r) a laboratory table.

13. The appliance according to claim 12 in a form of a moving handrail of an escalator or a moving walkway, wherein
a) the plasma source is integrated into the moving handrail, so that the plasma source moves with the handrail, or
b) the plasma source is stationary and arranged close to the surface of the handrail, so that the non-thermal plasma generated by the plasma source at least partially disinfects or sterilizes the surface of the handrail.

14. The appliance according to claim 12, in a form of a deodorant device comprising a rotatable ball for applying a deodorant, wherein
a) the plasma source is integrated into the rotatable ball, or
b) the plasma source is arranged stationary outside the rotatable ball.

15. The appliance according to claim 12, in a form of a deodorant device comprising an applicator for applying a deodorant comprising at least one nozzle for spraying an agent onto the surface, wherein the agent interacts with the non-thermal plasma thereby improving a disinfecting or sterilizing, effect of the non-thermal plasma.

16. The appliance according to claim 12, in a form of a deodorant device, wherein the deodorant device does not comprise any movable parts and relies solely on the disinfecting or sterilizing effect of the plasma.

17. The appliance according to 12, in a form of a deodorant device comprising a front end, the front end comprising a first outer electrode of the plasma source and a second inner electrode of the plasma source.

18. The appliance according to claim 17, wherein the front end-has a convex shape.

19. The appliance according to claim 17, wherein the front end comprises a non-conductive material.

20. The appliance according to claim 17, wherein the first outer electrode and/or the second inner electrode are/is realized by deposition of a conducting substance on an inner and/or outer surface of the front end, wherein the conductive substance is a metal, a conducting plastic or a conducting glue.

21. The appliance according to claim 20, wherein the front end and the first and/or second electrodes are realized by injection molding different materials, wherein a conductive material is used for the first and/or second electrodes and non-conductive material is used for the front end.

22. The appliance according to claim 17, wherein the first outer electrode and/or the second inner electrode;
i) a) are finger-shaped intertwining each other from opposite directions, or
b) are spiral-shaped intertwining each other, and/or
c) comprise interlocking branches or kinks or
ii) a) are arranged in separate adjacent electrode layers, and/or
b) each of the electrode layers is planar and the separate electrode layers are arranged coplanar relative to each other, and/or
c) the electrodes are switched at a constant or variable frequency.

23. The appliance according to claim 17, wherein an inner and/or outer surface of the front end comprise/comprises a number of grooves receiving a conductive material to realize the first outer electrode and/or the second inner electrode.

24. The appliance according to claim 17, wherein the front end is a removable cap.

25. The appliance according to claim 24, wherein a number of removable caps is provided having different embodiments of electrodes.

26. The appliance according to claim 12 in a form of a household appliance selected from the group consisting of a dishwasher and a dryer, wherein the plasma source is integrated in walls a housing of the household appliance.

27. The application according to claim 12, said appliance being a container comprising a body, a lid, and a counter electrode with a conductive area, wherein the lid comprises a first electrode and wherein the counter electrode is connected to an electric source.

28. The appliance according to claim 27, wherein the first electrode of the lid and/or the conductive area of the counter electrode comprises a conductive material.

29. The appliance according to claim 27, wherein the counter electrode is realized separately from the lid.

30. The appliance according to claim 27, wherein the counter electrode comprises apertures connected to a suction device, sucking air through the apertures.

31. The appliance according to claim 27, wherein the first electrode of the lid comprises strip-like conductive areas, arranged in a first pattern, and wherein the conductive area of the counter electrode is realized as a continuous area of conductive material.

32. The appliance according to claim 27, wherein the first electrode of the lid comprises a strip-like conductive area, arranged in a first pattern, and wherein the conductive area of the counter electrode is realized in a second pattern that is identical or substantially similar to the first pattern.

33. The appliance according to claim 27, wherein the first electrode of the lid comprises conductive strip-like areas, and realizes a first pattern, wherein the conductive sections are oriented in a first direction, and wherein the conductive area of the counter electrode comprises strip-like conductive sections and realizes a second pattern, wherein the conductive sections are oriented in a second direction, and wherein the strip-like areas of the first pattern and the strip-like area of the second pattern include an angle α chosen in a range of 0°<α<180°.

34. The appliance according to claim 1, wherein the pathogenic germs comprise
a) bacteria,
b) spores,
c) viruses,
d) fungi,
e) prions,
f) biofilms comprising any of the aforementioned pathogenic germs, and/or
g) microorganisms.
